(19) United States
(12) Patent Application Publication
(10) Pub. No.: US 2026/0041713 A1
(43) Pub. Date: Feb. 12, 2026

(54) ENHANCED SKIN REGENERATION AND THERAPEUTIC DELIVERY USING DIAMOND-AUGMENTED ZINC OXIDE

(71) Applicant: B.A.I. Biosciences, Inc., CAMBRIDGE, MA (US)

(72) Inventor: Xuefei Bai, Cambridge, MA (US)

(21) Appl. No.: 19/297,952

(22) Filed: Aug. 12, 2025

Related U.S. Application Data
(60) Provisional application No. 63/682,277, filed on Aug. 12, 2024.

(51) Int. Cl. A61K 33/30; A61K 9/00; A61K 9/14; A61K 33/44; A61K 41/00; A61P 17/02

(52) U.S. Cl. CPC A61K 33/30; A61K 9/0014; A61K 9/14; A61K 33/44; A61K 41/0057; A61P 17/02

(57) ABSTRACT

Disclosed herein are methods of enhancing skin regeneration, promoting wound healing (e.g., wherein the wound is a burn or actinic keratosis), and/or enhancing the topical delivery of active agents, the methods comprising the steps of applying a topical composition, comprising nanodiamond-zinc oxide core-shell particles, to the skin of a subject in need thereof.

### CROSS-REFERENCE TO RELATED APPLICATIONS

This application claims the benefit of U.S. Provisional Patent Application No. 63/682,277, filed Aug. 12, 2024, the contents of which are herein incorporated by reference in their entirety for all purposes.

### BACKGROUND OF THE DISCLOSURE

Recent advancements in dermatological therapeutics have increasingly emphasized holistic approaches that not only protect but also rejuvenate the skin. There is a growing demand for innovative treatments that enhance skin regeneration and promote healing, especially following cosmetic procedures or due to environmental damage. Traditional skincare solutions often fall short in providing these comprehensive benefits, underscoring the need for formulations that address these multifaceted requirements.

Conventional skin care ingredients are insufficient at addressing these holistic approaches in view of their limitations, such as poor absorption and limited efficacy in promoting skin healing and regeneration. Accordingly, there is a need in the art skincare products that enhance skin regeneration, promote healing, and/or enhance the topical delivery of active agents.

### SUMMARY OF THE DISCLOSURE

In one aspect, the present disclosure provides a method of promoting wound healing to a subject in need thereof comprising the step of applying a topical composition to the subject's skin comprising a wound, wherein the topical composition comprises particles with a core-shell structure, wherein the core comprises a nanodiamond and the shell comprises zinc oxide. In some embodiments, the wound healing is promoted by dermal fibroblast migration or growth. In some embodiments, the wound healing is promoted by human dermal fibroblast migration or growth.

In one aspect, the present disclosure provides a method of promoting dermal fibroblast migration or growth in a subject comprising the step of applying a topical composition to the subject's skin, wherein the topical composition comprises particles with a core-shell structure, wherein the core comprises a nanodiamond and the shell comprises zinc oxide. In some embodiments, the skin comprises a wound and the dermal fibroblast migration promotes wound healing. In some embodiments, the dermal fibroblast is a human dermal fibroblast.

In some embodiments of any of the above aspects, the wound is a burn. In some embodiments, the burn is a sunburn. In some embodiments, the burn is from a laser. In some embodiments, the burn is from cryotherapy. In some embodiments of any of the above aspects, the wound is an actinic keratosis. In some embodiments of any of the above aspects, the wound is a scratch, incision or a laceration.

In one aspect, the present disclosure provides a method of treating actinic keratosis in a subject in need thereof comprising the step of applying a topical composition to the subject's skin comprising a wound, wherein the topical composition comprises particles with a core-shell structure, wherein the core comprises a nanodiamond and the shell comprises zinc oxide.

In one aspect, the present disclosure provides a method of promoting collagen production in a subject comprising the step of applying a topical composition to the subject's skin, wherein the topical composition comprises particles with a core-shell structure, wherein the core comprises a nanodiamond and the shell comprises zinc oxide.

In some embodiments, the particles stimulate collagen production in dermal fibroblast cells. In some embodiments, the dermal fibroblast cells are human dermal fibroblast cells. In some embodiments, the human dermal fibroblast cells are aged human dermal fibroblast cells. In some embodiments, the collagen production is promoted after exposure of the subject's skin to ultraviolet (UV) radiation. In some embodiments, the UV radiation is UVA radiation. In some embodiments, the topical composition is applied at night.

In one aspect, the present disclosure provides a method of reducing collagen degradation in a subject, comprising the step of applying a topical composition to the subject's skin, wherein the topical composition comprises particles with a core-shell structure, wherein the core comprises a nanodiamond and the shell comprises zinc oxide. In some embodiments, the collagen degradation is age-associated collagen degradation. In some embodiments, the collagen degradation is caused by exposure of the subject's skin to ultraviolet (UV) radiation. In some embodiments, the UV radiation is UVA radiation.

In one aspect, the present disclosure provides a method of enhancing transdermal delivery of an active agent to a subject, comprising the step of applying a topical composition to the subject's skin, wherein the topical composition comprises the active agent and particles with a core-shell structure, wherein the core comprises a nanodiamond and the shell comprises zinc oxide. In some embodiments, the active agent is niacinamide. In some embodiments, the topical composition comprises between 2% and 20% niacinamide. In some embodiments, the topical composition comprises 5% niacinamide.

In some embodiments of any of the above aspects, the topical composition further comprises at least one topically acceptable carrier.

In some embodiments of any of the above aspects, the topical composition comprises 0.01 to 30 percent by weight of the particles. In some embodiments of any of the above aspects, the topical composition comprises between 1 μg/mL and 100 μg/mL of the particles. In some embodiments of any of the above aspects, the topical composition comprises 10 μg/mL of the particles.

In some embodiments of any of the above aspects, the core of the particle is partially or fully encapsulated by, embedded in, or attached to the shell of the particle. In some embodiments of any of the above aspects, the core and the shell form a particle through covalent bonds, non-covalent bonds or encapsulation of the core by the shell. In some embodiments of any of the above aspects, the core and the shell are in direct contact.

In some embodiments of any of the above aspects, the core comprises a nanodiamond having a diameter between 3 nm and 50 nm. In some embodiments of any of the above aspects, the core comprises a nanodiamond having a diameter between 3 nm and 10 nm.

In some embodiments of any of the above aspects, the particles have a diameter of at least 100 nm. In some embodiments of any of the above aspects, the particles have a Z-average diameter between 250 nm and 1000 nm. In some embodiments of any of the above aspects, the particles have an approximate Z-average diameter of 500 nm.

In some embodiments of any of the above aspects, the subject is a human subject. In some embodiments, the human subject is at least 25 years old.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-1D demonstrate the size uniformity, the porous nature, and the functional characteristics of ND-ZnO particles used in the claimed methods. FIG. 1A provides transmission electron microscopy (TEM) images of ND-ZnO particles, displaying a porous sphere composed of clustered zinc oxide crystallites surrounding a central nanodiamond core. Scale bars: left image 100 nm, right image 20 nm. FIG. 1B provides scanning electron microscopy (SEM) image of ND-ZnO particles (top left) and corresponding elemental mapping images showing the distribution of Zinc (top right, “Zn,” green), Carbon (bottom left, “C,” purple), and Oxygen (bottom right, “O,” red). Scale bars: 500 nm. FIG. 1C provides UV absorption spectra comparing an SPF 30 sunscreen containing ND-ZnO (blue lines) with two commercially available sunscreens: Sunscreen A (SPF 50, containing 11% ZnO, pink lines) and Sunscreen B (SPF 50, containing 10% ZnO and tridecyl salicylate, green lines). Solid lines represent absorption at 1.3 mg/cm², and dotted lines represent absorption at 0.5 mg/cm². The characteristic ZnO absorption peak around 370 nm is most prominent in the ND-ZnO formulation. The top solid line and top dashed line, as the Y-axis (i.e., at a wavelength of 290 nm) are for the SPF 30 sunscreen containing ND-ZnO. The middle solid line and middle dashed line, as the Y-axis (i.e., at a wavelength of 290 nm) are for Sunscreen B. The bottom solid line and bottom dashed line, as the Y-axis (i.e., at a wavelength of 290 nm) are for Sunscreen A.

FIG. 1D provides a reactive oxygen species (ROS) generation index measured over 6 hours using the DCFH-DA method during prolonged irradiation with a solar simulator. ND-ZnO generated significantly less ROS compared to conventional ZnO. The top line is for Conventional ZnO, and the bottom line is for ND-ZnO.

FIGS. 2A-2D demonstrate that ND-ZnO particles promote wound healing. FIG. 2A provides the quantification of cell migration in a wound healing assay. ND-ZnO particles significantly increase cell migration compared to the control, demonstrating 198% more human dermal fibroblast growth and migration. FIG. 2B provides fluorescence microscopy images of wound healing assays at 0 and 24 hours. The top panels show control samples, while the bottom panels show samples treated with ND-ZnO particles. Green fluorescence indicates human dermal fibroblasts. The images illustrate the enhanced wound closure and fibroblast migration in the ND-ZnO-treated samples compared to the control. FIG. 2C provides clinical study images of patient healing post ablative CO₂-laser treatment. Comparison of wound healing progression over 4 days shows that ND-ZnO treatment results in faster and more effective healing compared to the standard of care (Aquaphor). FIG. 1D provides clinical study images showing a patient who had actinic keratoses removed. The patient, who had been suffering from persistent post-procedure burn, experienced notable healing after using cream comprising ND-ZnO cream for just one week.

FIGS. 3A-3B demonstrates that ND-ZnO particles improve total collagen production and prevents collagen degradation. FIG. 3A demonstrates the total collagen production of human dermal fibroblasts post-treatment with UVA. The graph shows collagen production levels in negative control (no treatment), positive control (100 μg/mL vitamin C+7 μg/mL vitamin E), and ND-ZnO (10 μg/mL). FIG. 3B demonstrates the total collagen production of human dermal fibroblasts pre-treatment with UVA. The graph shows collagen production levels in negative control (no treatment), positive control (100 μg/mL vitamin C+7 μg/mL vitamin E), and ND-ZnO (10 μg/mL).

FIG. 4 provides the relative niacinamide skin absorption (%) over time (hours) comparing a 5% niacinamide solution with a ND-ZnO+5% niacinamide solution. The ND-ZnO containing cream demonstrated over 2× greater skin absorption of niacinamide than the niacinamide solution alone. The top line is for niacinamide solution with ND-ZnO containing cream, and the bottom line is for niacinamide solution.

FIG. 5 provides a diagram detailing the process when UV irradiation is absorbed by a core-shell nanodiamond doped zinc oxide particle. The UV irradiation generates an exciton, where an electron is excited from the valence band (VB) to the conduction band (CB) of the shell, leaving a hole. The hole migrates to the core-shell interface and transfers to the highest occupied molecular orbital (HOMO) of nanodiamond. The nanodiamond core scavenges the hole either by an internal oxidation process or by harvesting the electron in CB of the shell.

FIG. 6 provides a diagram detailing the process where a hole migrates to the core-shell interface and transfers to the HOMO of a nanodiamond.

### DETAILED DESCRIPTION OF THE DISCLOSURE

Unless otherwise defined herein, scientific and technical terms used in this application shall have the meanings that are commonly understood by those of ordinary skill in the art. In case of conflict, the present specification, including definitions, will control.

It should be understood that any of the embodiments described herein, including those described under different aspects of the disclosure and different parts of the specification (including embodiments described only in the Examples) can be combined with one or more other embodiments of this disclosure, unless explicitly disclaimed or improper. Combinations of embodiments are not limited to those specific combinations recited in the claims, including the dependent claims.

All of the publications, patents and published patent applications referred to in this application are specifically incorporated by reference in their entirety. In case of conflict, the present specification, including its specific definitions, will control.

Where aspects or embodiments are described in terms of a Markush group or other grouping of alternatives, the present application encompasses not only the entire group listed as a whole, but each member of the group individually and all possible subgroups of the main group, and also the main group absent one or more of the group members.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

Any example(s) following the term “e.g.,” or “for example” is not meant to be exhaustive or limiting.

Unless otherwise required by context, singular terms shall include pluralities and plural terms shall include the singular.

The articles “a,” “an” and “the” are used herein to refer to one or to more than one (i.e., to at least one) of the grammatical object of the article.

When a range of values is listed, it is intended to encompass each value and sub-range within the range.

As used herein, the terms “about” and “approximate” modifying the quantity of an ingredient, parameter, calculation, or measurement in the compositions employed in the methods of the disclosure refer to the variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within 10%, more typically still within 5%, of a given value or range. The terms “about” and “approximate” also encompass amounts that differ due to different equilibrium conditions for a composition resulting from a particular initial mixture. Whether or not modified by the terms “about” and “approximate,” the paragraphs include equivalents to the quantities. Reference to “about” a value or parameter or an “approximate” value or parameter herein includes (and describes) in particular embodiments that are directed to that value or parameter per se. For example, description referring to “about X” includes description of “X”, and description referring to “an approximate value of X” includes description of “a value of X.”

### Definitions

In order that the disclosure may be more readily understood, certain terms are first defined. These definitions should be read in light of the remainder of the disclosure, as understood by a person of ordinary skill in the art. Unless defined otherwise, all technical and scientific terms used herein have the same meaning as commonly understood by a person of ordinary skill in the art.

As used herein, the term “actinic keratosis” or “actinic keratoses” refers to a rough, scaly patch on the skin that develops from years of sun exposure.

As used herein, the term “active agent” refers to a compound that provides pharmacological activity or other direct effect in the diagnosis, cure, mitigation, treatment, or prevention of disease, or to affect the structure or any function of the body of the subject.

The term “aging” or its cognates (e.g., aged) as used herein is the time-related deterioration of the physiological functions necessary for survival and reproduction. Aging may be due to natural or unnatural causes. In some embodiments, aging results from the passage of time. In some embodiments, aging is accelerated by one or more environmental factors, e.g., exposure to UV radiation.

As used herein, the term “collagen production” refers to the natural process by which living cells, e.g., human dermal fibroblasts, synthesize collagen.

As used herein, the term “core-shell” or “core-shell structure” refers to a structure of a particle where a first component is partially or fully encapsulated by or embedded in a second component, such that no more than half of the first component's surface area is exposed. In some embodiments, none of the first component's surface area is exposed. As used herein, the first component is a core, and the second component is a shell. The core and the shell may form a particle through covalent bonds, non-covalent bonds, or encapsulation.

As used herein, the term “degraded” refers to a material or process that has a measurable decrease in its normal quality or function. For example, a degraded material or process may have a 5%, 10%, 15%, 20%, 25%, 30%, 35%, 40%, 45%, 50%, 55%, 60%, 65%, 70%, 75%, 80%, 85%, 90%, 95%, 99%, or less, or any range derivable therein, reduction of quality or function as compared to normal.

As used herein, the term “dermal fibroblasts” refers to cells within the dermis layer of skin, which are responsible for the generating connective tissue. In some embodiments, the dermal fibroblasts are human dermal fibroblasts (HDFs). In some embodiments, the connective tissue contains collagen.

As used herein, the term “growth” refers to the process by which cells, e.g., human dermal fibroblasts, proliferate. In some embodiments, human dermal fibroblast migration or growth is involved in wound healing.

The term “healing” or its cognates (e.g., to heal) as used herein, unless otherwise indicated, means eliminating, modifying, improving, lessening, reversing, alleviating, or inhibiting the progress of, or preventing a condition or disorder, or one or more symptoms of, or associated with, such condition or disorder of this disclosure. For example, as used herein, “wound healing” refers the process by which a tissue is partially or fully restored to its normal structure and/or function after an injury.

As used herein, the term “migration” refers to the process by which fibroblasts move across two-dimensional (2D) surfaces or three-dimensional (3D) tissues. Fibroblasts generally migrate across 2D surfaces by forming lamellipodia (actin-rich extensions at the leading edge of the cell) and across 3D tissues by forming pseudopodia (actin-filled structures that can be large and sheet-like or think and finger-like and may comprise multiple lamellae).

As used herein, the term “nanodiamond (ND)” refers to particles with diameters between 1-1000 nanometers comprising predominantly sp3 carbons. In some embodiments, the ND has a diameter between 3-50 nm. In some embodiments, the ND has a diameter between 3-10 nm.

The term “night” as used herein refers to the period of time when the sun is below the horizon.

As used herein, the term “promotion” refers to a material or process that has a measurable increase in its normal quality or function. For example, a promoted material or process may have a 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, increase of quality or function as compared to normal.

The terms “subject,” “individual,” or “patient” are used interchangeably herein and refer to a mammal. Mammals include, but are not limited to, farm animals (such as cows, horses, pigs, and goats), sport animals, pets (such as guinea pigs, cats, dogs, rabbits and horses), primates, mice and rats. In certain embodiments, a mammal is a human.

As used herein, the term “topical composition” refers to a composition that is intended for exterior bodily use. Topical compositions may be applied to the surface of the body, for example, on the skin.

As used herein, the term “topically acceptable” means that the item in question is compatible with a keratinous substrate. For example, a “topically acceptable carrier,” refers to compositions, carriers, diluents, and reagents which are compatible with application to a keratinous substrate such as skin, hair, scalp, or nails. The core-shell particles described herein can be mixed with excipients which are topically acceptable, compatible with the active ingredient, and in amounts suitable for the uses described herein. Non-limiting examples of topically acceptable carriers include glycerin, alcohols, organic solvents, fatty alcohols, fatty ethers, fatty esters, polyols, glycols, vegetable oils, mineral oils, liposomes, laminar lipid materials, water, and combinations thereof. Suitable topically acceptable carriers are well known in the art. See, e.g., The International Cosmetic Ingredient Dictionary and Handbook, 16^{th} Edition, 2016, Nikitakis, J. and Lange, B. Eds., Personal Care Products Council, Washington D.C.

As used herein, the term “ultraviolet (UV) radiation” refers to electromagnetic radiation, or light, that has an approximate wavelength of 10 to 400 nm. UVA radiation has a wavelength of 315-400 nm. UVB has a wavelength of 280-315 nm.

The term “wound” as used herein refers to any disruption of or damage to living tissue. Living tissue as used herein includes, but is not limited to, skin. In some embodiments, the wound is an abrasion, laceration, puncture, incision, or burn. In some embodiments, the burn can be a result of exposure to the sun, laser therapy, or cryotherapy. In some embodiments, the laser therapy is ablative CO₂-laser treatment. In some embodiments, the wound is an actinic keratosis.

As used herein, the term “Z-average” refers to the intensity weighted mean hydrodynamic size of the ensemble collection of particles measured by dynamic light scattering (DLS). As used herein, the Z-average is derived from a Cumulants analysis of the measured correlation curve, wherein a single particle size is assumed, and a single exponential fit is applied to the autocorrelation function.

### Particles

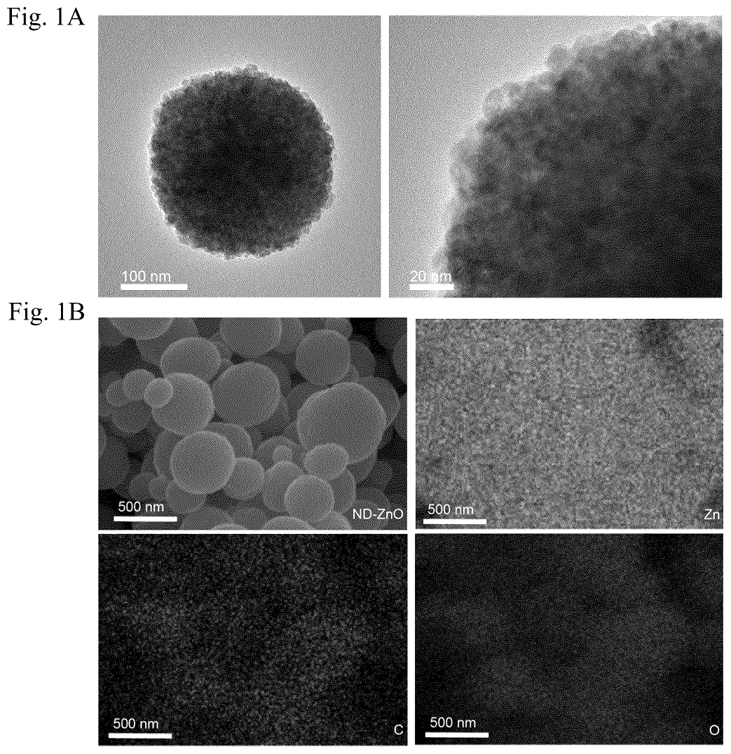

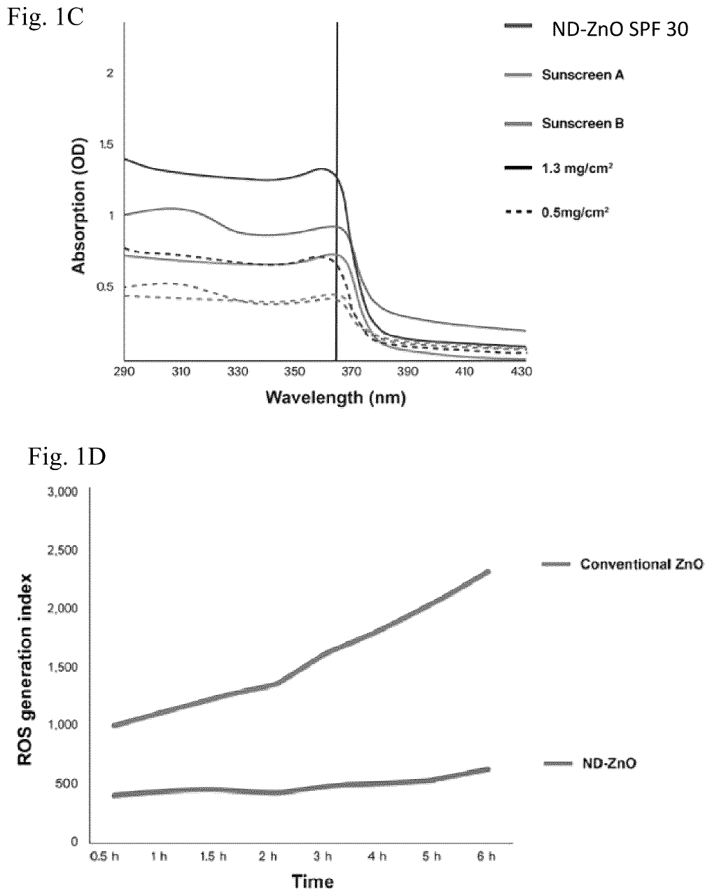

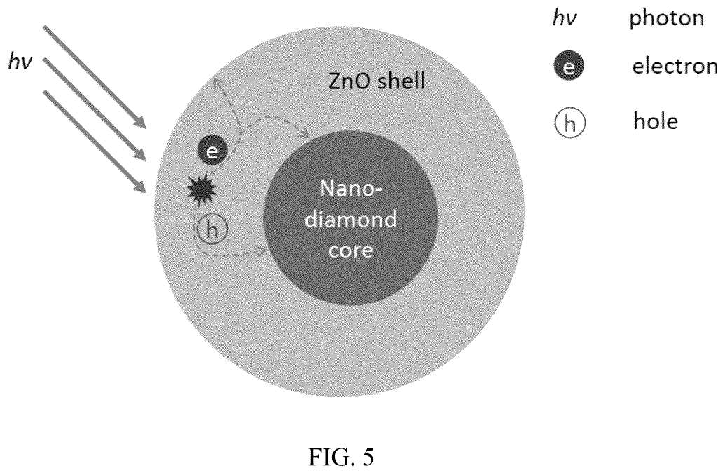

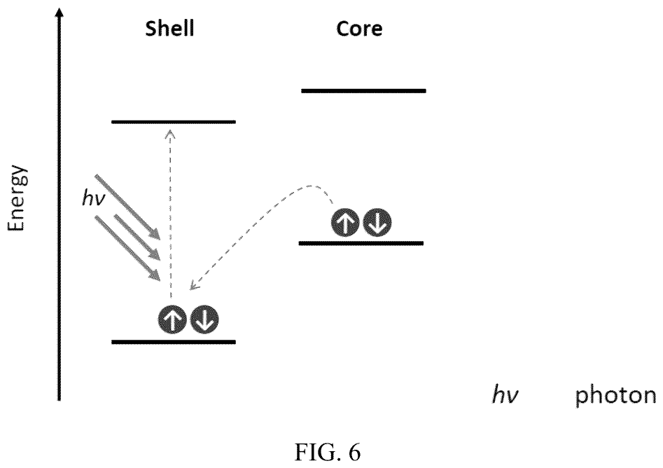

The Diamond-Augmented Zinc Oxide (ND-ZnO) technology disclosed herein represents a significant breakthrough in the field of dermatological therapeutics. The structure of ND-ZnO, comprises a porous sphere of zinc oxide crystallites surrounding a central nanodiamond core and offers distinct advantages in promoting skin health and regeneration. The properties, size, uniformity, and functional characteristics of ND-ZnO versus conventional ZnO preparations contribute to these advantages and are summarized in FIGS. 1A-1D, 5 and 6, as well as US Publication US2022/0071856A1, which is incorporated herein by reference in its entirety.

Transmission Electron Microscopy (TEM) images illustrate the porous structure and the central nanodiamond core, providing a clear view of the intricate design of these particles (FIG. 1a). Scanning Electron Microscopy (SEM) and elemental mapping images further reveal the uniform distribution of Zinc, Carbon, and Oxygen in ND-ZnO particles, confirming the integrity and consistency of the particle composition (FIG. 1B). This meticulous construction not only enhances the mechanical properties of ND-ZnO particles but also improves their functional capabilities in dermatological applications. Additionally, FIG. 1C shows that ND-ZnO particles exhibit superior UV-Vis absorption compared to conventional ZnO sunscreens. This enhanced performance is crucial for providing better protection against harmful UV radiation. FIG. 1D highlights ND-ZnO particles' significantly lower reactive oxygen species (ROS) generation during prolonged irradiation, up to 80% less than conventional ZnO. These properties underscore ND-ZnO particles' potential in reducing oxidative stress and preventing skin damage.

The methods disclosed herein utilize particles comprising a nanodiamond (ND) core and a zinc oxide (ZnO) shell. In some embodiments, the core of the particle is partially or fully encapsulated by, embedded in, or attached to the shell of the particle. In some embodiments, the core of the particle is fully encapsulated by, embedded in, or attached to the shell of the particle. In some embodiments, the core and the shell form a particle through covalent bonds, non-covalent bonds or encapsulation of the core by the shell. In some embodiments, the core and the shell are in direct contact.

In some embodiments, the particles have a diameter of at least 80 nm. In some embodiments, the particles have a diameter of at least 100 nm. In some embodiments, the particles have a diameter of at least 150 nm. In some embodiments, the particles have a diameter of at least 200 nm. In some embodiments, the particles have a diameter of at least 250 nm.

In some embodiments, the particles have a diameter of no more than 1500 nm. In some embodiments, the particles have a diameter of no more than 1400 nm. In some embodiments, the particles have a diameter of no more than 1300 nm. In some embodiments, the particles have a diameter of no more than 1250 nm. In some embodiments, the particles have a diameter of no more than 1200 nm. In some embodiments, the particles have a diameter of no more than 1100 nm. In some embodiments, the particles have a diameter of no more than 1000 nm.

In some embodiments, the particles have a diameter between 100 to 1500 nm. In some embodiments, the particles have a diameter between 100 to 1400 nm. In some embodiments, the particles have a diameter between 100 to 1386 nm. In some embodiments, the particles have a diameter between 100 to 1000 nm. In some embodiments, the particles have a diameter between 100 to 750 nm. In some embodiments, the particles have a diameter between 100 to 500 nm. In some embodiments, the particles have a diameter between 100 to 400 nm. In some embodiments, the particles have a diameter between 100 to 300 nm. In some embodiments, the particles have a diameter between 100 to 250 nm. In some embodiments, the particles have a diameter between 100 to 200 nm. In some embodiments, the particles have a diameter between 100 to 150 nm. In some embodiments, the particles have a diameter between 250 to 1400 nm. In some embodiments, the particles have a diameter between 250 to 1386 nm. In some embodiments, the particles have a diameter between 250 to 1000 nm. In some embodiments, the particles have a diameter between 250 to 750 nm. In some embodiments, the particles have a diameter between 250 to 500 nm. In some embodiments, the particles have a diameter between 250 to 400 nm. In some embodiments, the particles have a diameter between 250 to 300 nm. In some embodiments, the particles have a diameter between 250 to 290 nm. In some embodiments, the particles have a diameter between 290 to 1386 nm. In some embodiments, the particles have a diameter of 1500 nm. In some embodiments, the particles have a diameter of 1400 nm. In some embodiments, the particles have a diameter of 1386 nm. In some embodiments, the particles have a diameter of 1250 nm. In some embodiments, the particles have a diameter of 1000 nm. In some embodiments, the particles have a diameter of 750 nm. In some embodiments, the particles have a diameter of 500 nm. In some embodiments, the particles have a diameter of 400 nm. In some embodiments, the particles have a diameter of 300 nm. In some embodiments, the particles have a diameter of 290 nm. In some embodiments, the particles have a diameter of 250 nm. In some embodiments, the particles have a diameter of 200 nm. In some embodiments, the particles have a diameter of 150 nm.

In some embodiments, the particles have an approximate Z-average diameter of 100 to 1500 nm. In some embodiments, the particles have an approximate Z-average diameter of 100 to 1400 nm. In some embodiments, the particles have an approximate Z-average diameter of 100 to 1386 nm. In some embodiments, the particles have an approximate Z-average diameter of 100 to 1000 nm. In some embodiments, the particles have an approximate Z-average diameter of 100 to 750 nm. In some embodiments, the particles have an approximate Z-average diameter of 100 to 500 nm. In some embodiments, the particles have an approximate Z-average diameter of 100 to 400 nm. In some embodiments, the particles have an approximate Z-average diameter of 100 to 300 nm. In some embodiments, the particles have an approximate Z-average diameter of 100 to 250 nm. In some embodiments, the particles have an approximate Z-average diameter of 100 to 200 nm. In some embodiments, the particles have an approximate Z-average diameter of 100 to 150 nm. In some embodiments, the particles have an approximate Z-average diameter of 250 to 1400 nm. In some embodiments, the particles have an approximate Z-average diameter of 250 to 1386 nm. In some embodiments, the particles have an approximate Z-average diameter of 250 to 1000 nm. In some embodiments, the particles have an approximate Z-average diameter of 250 to 750 nm. In some embodiments, the particles have an approximate Z-average diameter of 250 to 500 nm. In some embodiments, the particles have an approximate Z-average diameter of 250 to 400 nm. In some embodiments, the particles have an approximate Z-average diameter of 250 to 300 nm. In some embodiments, the particles have an approximate Z-average diameter of 250 to 290 nm. In some embodiments, the particles have an approximate Z-average diameter of 290 to 1386 nm. In some embodiments, the particles have an approximate Z-average diameter of 1500 nm. In some embodiments, the particles have an approximate Z-average diameter of 1400 nm. In some embodiments, the particles have an approximate Z-average diameter of 1386 nm. In some embodiments, the particles have an approximate Z-average diameter of 1250 nm. In some embodiments, the particles have an approximate Z-average diameter of 1000 nm. In some embodiments, the particles have an approximate Z-average diameter of 750 nm. In some embodiments, the particles have an approximate Z-average diameter of 500 nm. In some embodiments, the particles have an approximate Z-average diameter of 400 nm. In some embodiments, the particles have an approximate Z-average diameter of 300 nm. In some embodiments, the particles have an approximate Z-average diameter of 290 nm. In some embodiments, the particles have an approximate Z-average diameter of 250 nm. In some embodiments, the particles have an approximate Z-average diameter of 200 nm. In some embodiments, the particles have an approximate Z-average diameter of 150 nm.

In some embodiments, the particles have a Z-average diameter of 100 to 1500 nm. In some embodiments, the particles have a Z-average diameter of 100 to 1400 nm. In some embodiments, the particles have a Z-average diameter of 100 to 1386 nm. In some embodiments, the particles have a Z-average diameter of 100 to 1000 nm. In some embodiments, the particles have a Z-average diameter of 100 to 750 nm. In some embodiments, the particles have a Z-average diameter of 100 to 500 nm. In some embodiments, the particles have a Z-average diameter of 100 to 400 nm. In some embodiments, the particles have a Z-average diameter of 100 to 300 nm. In some embodiments, the particles have a Z-average diameter of 100 to 250 nm. In some embodiments, the particles have a Z-average diameter of 100 to 200 nm. In some embodiments, the particles have a Z-average diameter of 100 to 150 nm. In some embodiments, the particles have a Z-average diameter of 250 to 1400 nm. In some embodiments, the particles have a Z-average diameter of 250 to 1386 nm. In some embodiments, the particles have a Z-average diameter of 250 to 1000 nm. In some embodiments, the particles have a Z-average diameter of 250 to 750 nm. In some embodiments, the particles have a Z-average diameter of 250 to 500 nm. In some embodiments, the particles have a Z-average diameter of 250 to 400 nm. In some embodiments, the particles have a Z-average diameter of 250 to 300 nm. In some embodiments, the particles have a Z-average diameter of 250 to 290 nm. In some embodiments, the particles have a Z-average diameter of 290 to 1386 nm. In some embodiments, the particles have a Z-average diameter of 1500 nm. In some embodiments, the particles have a Z-average diameter of 1400 nm. In some embodiments, the particles have a Z-average diameter of 1386 nm. In some embodiments, the particles have a Z-average diameter of 1250 nm. In some embodiments, the particles have a Z-average diameter of 1000 nm. In some embodiments, the particles have a Z-average diameter of 750 nm. In some embodiments, the particles have a Z-average diameter of 500 nm. In some embodiments, the particles have a Z-average diameter of 400 nm. In some embodiments, the particles have a Z-average diameter of 300 nm. In some embodiments, the particles have a Z-average diameter of 290 nm. In some embodiments, the particles have a Z-average diameter of 250 nm. In some embodiments, the particles have a Z-average diameter of 200 nm. In some embodiments, the particles have a Z-average diameter of 150 nm.

In some embodiments, the core of the particles comprises nanodiamonds having a diameter less than 60 nm. In some embodiments, the core of the particles comprises nanodiamonds having a diameter greater than 3 nm. In some embodiments, the core of the particles comprises nanodiamonds having a diameter between 3 nm and 50 nm. In some embodiments, the core of the particles comprises nanodiamonds having a diameter between 3 nm and 25 nm. In some embodiments, the core of the particles comprises nanodiamonds having a diameter between 3 nm and 20 nm. In some embodiments, the core of the particles comprises nanodiamonds having a diameter between 3 nm and 15 nm. In some embodiments, the core of the particles comprises nanodiamonds having a diameter between 3 nm and 10 nm.

In some embodiments, the shell of the particles has an average shell thickness greater than 100 nm. In some embodiments, the shell of the particles has an average shell thickness less than 700 nm. In some embodiments, the shell of the particles has an average shell thickness of 695 nm. In some embodiments, the shell of the particles has an average shell thickness of 500 nm. In some embodiments, the shell of the particles has an average shell thickness of 495 nm. In some embodiments, the shell of the particles has an average shell thickness of 250 nm. In some embodiments, the shell of the particles has an average shell thickness of 245 nm. In some embodiments, the shell of the particles has an average shell thickness of 145 nm. In some embodiments, the shell of the particles has an average shell thickness of 140 nm. In some embodiments, the shell of the particles has an average shell thickness of 125 nm. In some embodiments, the shell of the particles has an average shell thickness of 120 nm. In some embodiments, the shell of the particles has an average shell thickness of about 695 nm. In some embodiments, the shell of the particles has an average shell thickness of about 500 nm. In some embodiments, the shell of the particles has an average shell thickness of about 495 nm. In some embodiments, the shell of the particles has an average shell thickness of about 250 nm. In some embodiments, the shell of the particles has an average shell thickness of about 245 nm. In some embodiments, the shell of the particles has an average shell thickness of about 145 nm. In some embodiments, the shell of the particles has an average shell thickness of about 140 nm. In some embodiments, the shell of the particles has an average shell thickness of about 125 nm. In some embodiments, the shell of the particles has an average shell thickness of about 120 nm.

In some embodiments, the shell of the particles has an average shell thickness of 110 nm to 700 nm. In some embodiments, the shell of the particles has an average shell thickness of 110 nm to 695 nm. In some embodiments, the shell of the particles has an average shell thickness of 110 nm to 500 nm. In some embodiments, the shell of the particles has an average shell thickness of 110 nm to 495 nm. In some embodiments, the shell of the particles has an average shell thickness of 110 nm to 250 nm. In some embodiments, the shell of the particles has an average shell thickness of 110 nm to 245 nm. In some embodiments, the shell of the particles has an average shell thickness of 120 nm to 700 nm. In some embodiments, the shell of the particles has an average shell thickness of 120 nm to 695 nm. In some embodiments, the shell of the particles has an average shell thickness of 120 nm to 500 nm. In some embodiments, the shell of the particles has an average shell thickness of 120 nm to 495 nm. In some embodiments, the shell of the particles has an average shell thickness of 120 nm to 250 nm. In some embodiments, the shell of the particles has an average shell thickness of 120 nm to 245 nm. In some embodiments, the shell of the particles has an average shell thickness of 125 nm to 700 nm. In some embodiments, the shell of the particles has an average shell thickness of 125 nm to 695 nm. In some embodiments, the shell of the particles has an average shell thickness of 125 nm to 500 nm. In some embodiments, the shell of the particles has an average shell thickness of 125 nm to 495 nm. In some embodiments, the shell of the particles has an average shell thickness of 125 nm to 250 nm. In some embodiments, the shell of the particles has an average shell thickness of 125 nm to 245 nm. In some embodiments, the shell of the particles has an average shell thickness of 140 nm to 700 nm. In some embodiments, the shell of the particles has an average shell thickness of 140 nm to 695 nm. In some embodiments, the shell of the particles has an average shell thickness of 140 nm to 495 nm. In some embodiments, the shell of the particles has an average shell thickness of 140 nm to 500 nm. In some embodiments, the shell of the particles has an average shell thickness of 140 nm to 250 nm. In some embodiments, the shell of the particles has an average shell thickness of 140 nm to 245 nm. In some embodiments, the shell of the particles has an average shell thickness of 145 nm to 700 nm. In some embodiments, the shell of the particles has an average shell thickness of 145 nm to 695 nm. In some embodiments, the shell of the particles has an average shell thickness of 145 nm to 500 nm. In some embodiments, the shell of the particles has an average shell thickness of 145 nm to 495 nm. In some embodiments, the shell of the particles has an average shell thickness of 145 nm to 250 nm. In some embodiments, the shell of the has an average shell thickness of 145 nm to 245 nm.

In some embodiments, the shell of the particles has a shell thickness greater than 100 nm. In some embodiments, the shell of the particles has a shell thickness less than 700 nm. In some embodiments, the shell of the particles has a shell thickness of 695 nm. In some embodiments, the shell of the particles has a shell thickness of 500 nm. In some embodiments, the shell of the particles has a shell thickness of 495 nm. In some embodiments, the shell of the particles has a shell thickness of 250 nm. In some embodiments, the shell of the particles has a shell thickness of 245 nm. In some embodiments, the shell of the particles has a shell thickness of 145 nm. In some embodiments, the shell of the particles has a shell thickness of 140 nm. In some embodiments, the shell of the particles has a shell thickness of 125 nm. In some embodiments, the shell of the particles has a shell thickness of 120 nm. In some embodiments, the shell of the particles has a shell thickness of about 695 nm. In some embodiments, the shell of the particles has a shell thickness of about 500 nm. In some embodiments, the shell of the particles has a shell thickness of about 495 nm. In some embodiments, the shell of the particles has a shell thickness of about 250 nm. In some embodiments, the shell of the particles has a shell thickness of about 245 nm. In some embodiments, the shell of the particles has a shell thickness of about 145 nm. In some embodiments, the shell of the particles has a shell thickness of about 140 nm. In some embodiments, the shell of the particles has a shell thickness of about 125 nm. In some embodiments, the shell of the particles has a shell thickness of about 120 nm.

In some embodiments, the shell of the particles has a shell thickness of 110 nm to 700 nm. In some embodiments, the shell of the particles has a shell thickness of 110 nm to 695 nm. In some embodiments, the shell of the particles has a shell thickness of 110 nm to 500 nm. In some embodiments, the shell of the particles has a shell thickness of 110 nm to 495 nm. In some embodiments, the shell of the particles has a shell thickness of 110 nm to 250 nm. In some embodiments, the shell of the particles has a shell thickness of 110 nm to 245 nm. In some embodiments, the shell of the particles has a shell thickness of 120 nm to 700 nm. In some embodiments, the shell of the particles has a shell thickness of 120 nm to 695 nm. In some embodiments, the shell of the particles has a shell thickness of 120 nm to 500 nm. In some embodiments, the shell of the particles has a shell thickness of 120 nm to 495 nm. In some embodiments, the shell of the particles has a shell thickness of 120 nm to 250 nm. In some embodiments, the shell of the particles has a shell thickness of 120 nm to 245 nm. In some embodiments, the shell of the particles has a shell thickness of 125 nm to 700 nm. In some embodiments, the shell of the particles has a shell thickness of 125 nm to 695 nm. In some embodiments, the shell of the particles has a shell thickness of 125 nm to 500 nm. In some embodiments, the shell of the particles has a shell thickness of 125 nm to 495 nm. In some embodiments, the shell of the particles has a shell thickness of 125 nm to 250 nm. In some embodiments, the shell of the particles has a shell thickness of 125 nm to 245 nm. In some embodiments, the shell of the particles has a shell thickness of 140 nm to 700 nm. In some embodiments, the shell of the particles has a shell thickness of 140 nm to 695 nm. In some embodiments, the shell of the particles has a shell thickness of 140 nm to 495 nm. In some embodiments, the shell of the particles has a shell thickness of 140 nm to 500 nm. In some embodiments, the shell of the particles has a shell thickness of 140 nm to 250 nm. In some embodiments, the shell of the particles has a shell thickness of 140 nm to 245 nm. In some embodiments, the shell of the particles has a shell thickness of 145 nm to 700 nm. In some embodiments, the shell of the particles has a shell thickness of 145 nm to 695 nm. In some embodiments, the shell of the particles has a shell thickness of 145 nm to 500 nm. In some embodiments, the shell of the particles has a shell thickness of 145 nm to 495 nm. In some embodiments, the shell of the particles has a shell thickness of 145 nm to 250 nm. In some embodiments, the shell of the has a shell thickness of 145 nm to 245 nm.

### Topical Compositions

In the methods disclosed here, the ND-ZnO particles may be provided in a topical composition. In some embodiments, the topical composition further comprises at least one topically acceptable carrier.

In some embodiments, the topical composition comprises between 1 μg/mL and 100 μg/mL of the particles. In some embodiments, the topical composition comprises between 1 μg/mL and 50 μg/mL of the particles. In some embodiments, the topical composition comprises between 1 μg/mL and 25 μg/mL of the particles. In some embodiments, the topical composition comprises between 1 μg/mL and 10 μg/mL of the particles. In some embodiments, the topical composition comprises between 5 μg/mL and 100 μg/mL of the particles. In some embodiments, the topical composition comprises between 5 μg/mL and 50 μg/mL of the particles. In some embodiments, the topical composition comprises between 5 μg/mL and 25 μg/mL of the particles. In some embodiments, the topical composition comprises between 5 μg/mL and 10 μg/mL of the particles. In some embodiments, the topical composition comprises between 10 μg/mL and 100 μg/mL of the particles. In some embodiments, the topical composition comprises between 10 μg/mL and 50 μg/mL of the particles. In some embodiments, the topical composition comprises between 10 μg/mL and 25 μg/mL of the particles. In some embodiments, the topical composition comprises 1 μg/mL of the particles. In some embodiments, the topical composition comprises 5 μg/mL of the particles. In some embodiments, the topical composition comprises 10 μg/mL of the particles. In some embodiments, the topical composition comprises 25 μg/mL of the particles. In some embodiments, the topical composition comprises 50 μg/mL of the particles. In some embodiments, the topical composition comprises 100 μg/mL of the particles.

In some embodiments, the topical composition comprises 0.01 to 30 percent by weight of the particles. In some embodiments, the topical composition comprises 1 to 30 percent by weight of the particles. In some embodiments, the topical composition comprises 4 to 28 percent by weight of the particles.

In some embodiments, the topical composition further comprises an active agent. In some embodiments, the topical composition comprises between 2% and 20% by weight of the active agent. In some embodiments, the topical composition comprises between 2% and 15% by weight of the active agent. In some embodiments, the topical composition comprises between 2% and 10% by weight of the active agent. In some embodiments, the topical composition comprises between 2% and 5% by weight of the active agent. In some embodiments, the topical composition comprises between 5% and 20% by weight of the active agent. In some embodiments, the topical composition comprises between 5% and 15% by weight of the active agent. In some embodiments, the topical composition comprises between 5% and 10% by weight of the active agent. In some embodiments, the topical composition comprises between 10% and 20% by weight of the active agent. In some embodiments, the topical composition comprises between 10% and 15% by weight of the active agent. In some embodiments, the topical composition comprises between 15% and 20% by weight of the active agent. In some embodiments, the topical composition comprises 2% by weight of the active agent. In some embodiments, the topical composition comprises 5% by weight of the active agent. In some embodiments, the topical composition comprises 10% by weight of the active agent. In some embodiments, the topical composition comprises 15% by weight of the active agent. In some embodiments, the topical composition comprises 20% by weight of the active agent. In some embodiments, the topical composition comprises about 2% by weight of the active agent. In some embodiments, the topical composition comprises about 5% by weight of the active agent. In some embodiments, the topical composition comprises about 10% by weight of the active agent. In some embodiments, the topical composition comprises about 15% by weight of the active agent. In some embodiments, the topical composition comprises about 20% by weight of the active agent.

In some embodiments, the active agent is niacinamide. In some embodiments, the topical composition comprises between 2% and 20% of niacinamide. In some embodiments, the topical composition comprises between 2% and 15% of niacinamide. In some embodiments, the topical composition comprises between 2% and 10% of niacinamide. In some embodiments, the topical composition comprises between 2% and 5% of niacinamide. In some embodiments, the topical composition comprises between 5% and 20% of niacinamide. In some embodiments, the topical composition comprises between 5% and 15% of niacinamide. In some embodiments, the topical composition comprises between 5% and 10% of niacinamide. In some embodiments, the topical composition comprises between 10% and 20% of niacinamide. In some embodiments, the topical composition comprises between 10% and 15% of niacinamide. In some embodiments, the topical composition comprises between 15% and 20% of niacinamide. In some embodiments, the topical composition comprises 2% of niacinamide. In some embodiments, the topical composition comprises 5% of niacinamide. In some embodiments, the topical composition comprises 10% of niacinamide. In some embodiments, the topical composition comprises 15% of niacinamide. In some embodiments, the topical composition comprises 20% of niacinamide. In some embodiments, the topical composition comprises about 2% of niacinamide. In some embodiments, the topical composition comprises about 5% of niacinamide. In some embodiments, the topical composition comprises about 10% of niacinamide. In some embodiments, the topical composition comprises about 15% of niacinamide. In some embodiments, the topical composition comprises about 20% of niacinamide.

### Methods of Use

To evaluate the efficacy of ND-ZnO particles in various skin regenerative processes, experiments focusing on wound healing, collagen synthesis, and the transdermal delivery of active ingredients were conducted. In the wound healing assays, ND-ZnO particles significantly promoted human dermal fibroblast growth and migration, resulting in a 198% increase in cell migration compared to the control. Collagen synthesis assays demonstrated that ND-ZnO particles effectively prevented and reversed age-associated collagen degradation, with significant improvements in collagen production. Additionally, permeation tests revealed that ND-ZnO particles significantly enhanced the skin absorption of niacinamide, achieving a 203% increase in absorption over a 12-hour period.

Clinical trials further validated these findings. Patients treated with ND-ZnO particles after CO₂-laser procedures exhibited faster and more effective healing, while a separate trial showed significant improvements in patients with actinic keratosis following ND-ZnO particle treatment.

These results collectively highlight the potential of ND-ZnO technology in dermatological therapeutics, offering enhanced wound healing, improved collagen synthesis, and better active ingredient delivery.

In one aspect, the present disclosure provides a method of promoting wound healing in a subject in need thereof comprising the step of applying a topical composition to the subject's skin, wherein the skin to which the topical compositions is applied comprises a wound, wherein the topical composition comprises ND-ZnO particles disclosed herein. Another aspect of the disclosure provides the use of ND-ZnO particles of the disclosure in the manufacture of a medicament for promoting wound healing on the skin of a subject. A further aspect of the disclosure provides the ND-ZnO particles of the disclosure for use in promoting wound healing on the skin of a subject. In some embodiments, the wound healing is promoted by dermal fibroblast migration or growth. In some embodiments, the wound healing is promoted by human dermal fibroblast migration or growth.

In another aspect, the present disclosure provides a method of promoting dermal fibroblast migration or growth in a subject comprising the step of applying a topical composition to the subject's skin, wherein the topical composition comprises the ND-ZnO particles of the disclosure with a core-shell structure. Another aspect of the disclosure provides the use of the ND-ZnO particles of the disclosure in the manufacture of a medicament for promoting dermal fibroblast migration or growth in a subject. A further aspect of the disclosure provides the ND-ZnO particles of the disclosure for use in promoting dermal fibroblast migration or growth in a subject. In some embodiments, the subject's skin comprises a wound and the dermal fibroblast migration promotes wound healing.

In some embodiments of any of the above aspects, the wound is a burn. In some embodiments, the burn is a sunburn, is from a laser or is from cryotherapy. In some embodiments, the burn is a sunburn. In some embodiments, the burn is from a laser. In some embodiments the burn is from cryotherapy.

In some embodiments of any of the above aspects, the wound is an actinic keratosis.

In some embodiments of any of the above aspects, the wound is a scratch, an incision, or a laceration. In some embodiments of any of the above aspects, the wound is a scratch. In some embodiments of any of the above aspects, the wound is an incision. In some embodiments of any of the above aspects, the wound is a laceration.

In another aspect, the present disclosure provides a method of treating actinic keratosis in a subject in need thereof comprising the step of applying a topical composition to the subject's skin comprising a wound, wherein the topical composition comprises the ND-ZnO particles of the disclosure. Another aspect of the disclosure provides the use of the ND-ZnO particles of the disclosure in the manufacture of a medicament for treating actinic keratosis in a subject in need thereof. A further aspect of the disclosure provides the ND-ZnO particles of the disclosure for use in treating actinic keratosis in a subject in need thereof.

In another aspect, the present disclosure provides a method of promoting collagen production in a subject comprising the step of applying a topical composition to the subject's skin, wherein the topical composition comprises the ND-ZnO particles of the disclosure. Another aspect of the disclosure provides the use of the ND-ZnO particles of the disclosure in the manufacture of a medicament for promoting collagen production in a subject. A further aspect of the disclosure provides the ND-ZnO particles of the disclosure for use in promoting collagen production in a subject. In some embodiments, the particles stimulation collagen production in dermal fibroblast cells. In some embodiments, the dermal fibroblast cells are aged dermal fibroblast cells. In some embodiments, the dermal fibroblast cells are human dermal fibroblast cells. In some embodiments, the human dermal fibroblast cells are aged human dermal fibroblast cells. In some embodiments, collagen production is promoted after exposure of the subject's skin to ultraviolet (UV) radiation. In some embodiments, the UV radiation is UVA radiation.

In another aspect, the present disclosure provides a method of reducing collagen degradation in a subject, comprising the step of applying a topical composition to the subject's skin, wherein the topical composition comprises the ND-ZnO particles of the disclosure. Another aspect of the disclosure provides the use of the ND-ZnO particles of the disclosure in the manufacture of a medicament for reducing collagen degradation in a subject. A further aspect of the disclosure provides the ND-ZnO particles of the disclosure for use in reducing collagen degradation in a subject. In some embodiments, collagen degradation is prevented. In some embodiments, the collagen degradation is age-associated collagen degradation. In some embodiments, the collagen degradation is caused by exposure of the subject's skin to ultraviolet (UV) radiation. In some embodiments, the UV radiation is UVA radiation.

In another aspect, the present disclosure provides a method of enhancing transdermal delivery of an active agent to a subject, comprising the step of applying a topical composition to the subject's skin, wherein the topical composition comprises the active agent and the ND-ZnO particles of the disclosure. Another aspect of the disclosure provides the use of the ND-ZnO particles of the disclosure in the manufacture of a medicament for enhancing transdermal delivery of an active agent to a subject. A further aspect of the disclosure provides the ND-ZnO particles of the disclosure for use in enhancing transdermal delivery of an active agent to a subject.

In some embodiments of any of the above aspect, the topical composition is applied to the subject's skin at night. In some embodiments of any of the above aspects, the topical composition is applied to the subject's skin indoors. In some embodiments of any of the above aspects, the subject is not be exposed to UV radiation. In some embodiments of any of the above aspects, the subject has minimal risk at exposure to UV radiation. In some embodiments of any of the above aspects, the topical composition is not being used as a sunscreen.

In some embodiments of any of the above aspects, the subject is a human subject. In some embodiments, the human subject is at least 25 years old. In some embodiments, the human subject is at least 30 years old. In some embodiments, the human subject is at least 40 years old. In some embodiments, the human subject is at least 50 years old. In some embodiments, the human subject is at least 60 years old.

### EXAMPLES

Exemplary methods and materials are described herein, although methods and materials similar or equivalent to those described herein can also be used in the practice or testing of the various aspects and embodiments of the disclosure. The materials, methods, and examples are illustrative only and not intended to be limiting.

### Example 1: ND-ZnO Particles Promote Human Dermal Fibroblast Migration and Growth

Human dermal fibroblasts (HDFs) were seeded at a concentration of 200,000 cells/well in a 24-well plate and allowed to incubate at 37° C., 5% CO₂ for 24 hours. A scratch was made in each well using a 200 μl pipette tip creating perpendicular scratch. Media was removed and cells were washed in PBS three times before adding ND-ZnO conditioned media (10 μL/mL) or control media.

Optical microscopy was employed to analyze five distinct regions along the scratches of each well at baseline and after 24 hours. The areas occupied by HDFs were quantified using Image Pro Plus software. The extent of area closure was then calculated and expressed as a percentage in comparison to control cells that remained untreated.

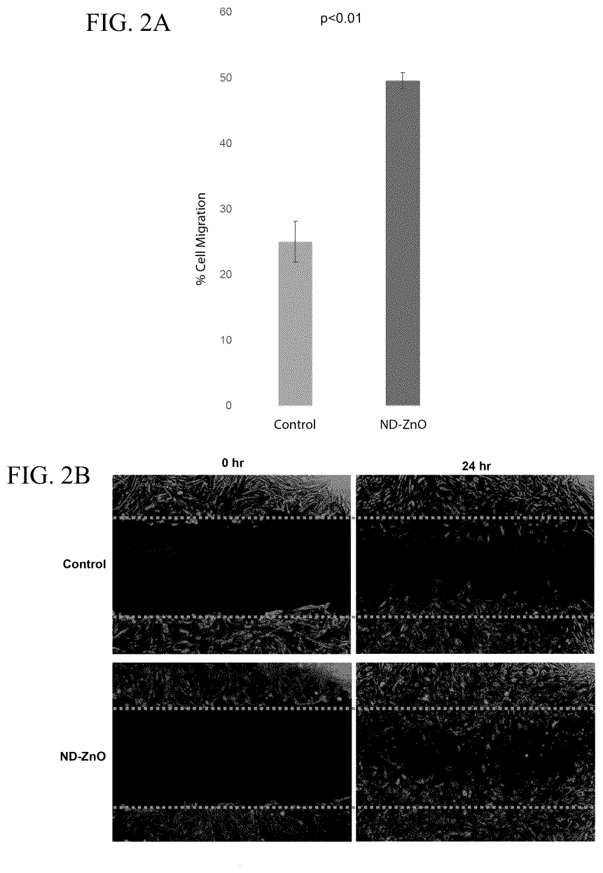

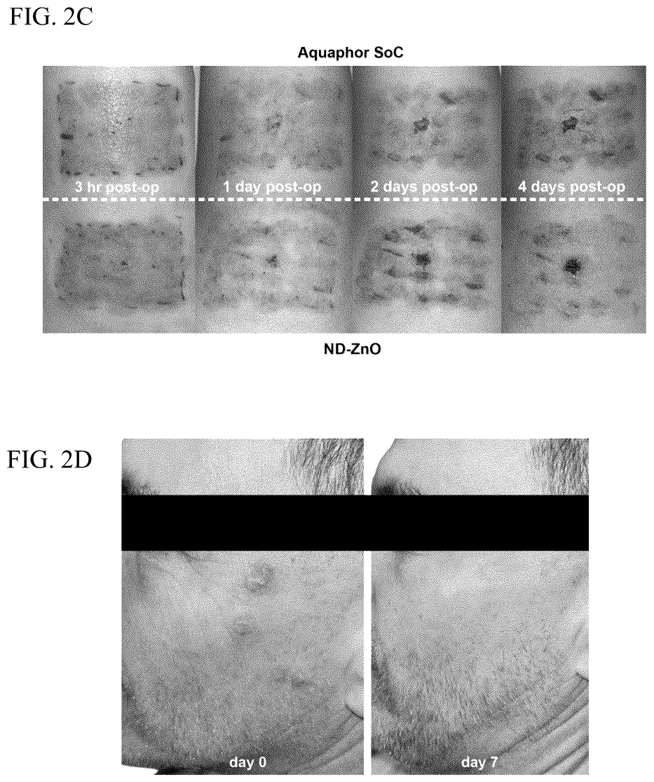

The comparative analysis revealed significant migration of HDFs treated with ND-ZnO, particles evaluated by quantifying the closure of the scratched area. Across all three plates assayed, the migration of HDFs treated with ND-ZnO particles was significantly greater than that of the control group. See FIG. 2A. On average, the wound closure observed in the control group was 25.05% (SD=3.14). In contrast, the ND-ZnO-treated HDFs demonstrated an average of 49.57% wound closure, which is 1.98 times the HDF proliferation of the control group, with lower variability (SD=1.23). The difference in HDF growth between the two conditions was statistically significant (p<0.01). This data underscores the potential of ND-ZnO particles as an effective agent for skin regeneration, promoting markedly greater HDF proliferation and migration over a 24-hour period than the control.

Fluorescence microscopy further corroborated these findings. At both the 0 and 24-hour marks, ND-ZnO treated samples displayed considerably more fibroblast activity and wound closure than the control samples. See FIG. 2B. The green fluorescence, indicative of human dermal fibroblasts, was markedly more pronounced in the treated samples, demonstrating the potent effect of ND-ZnO particles in accelerating cellular processes critical for wound repair.

Clinical studies provided additional evidence of the efficacy of ND-ZnO particles in clinical applications. Patients undergoing post ablative CO₂-laser treatment showed faster and more effective healing when treated with ND-ZnO compared to the standard of care (Aquaphor). See FIG. 2C. Over a span of four days, the ND-ZnO treated areas exhibited reduced redness and faster tissue regeneration, highlighting the potential of ND-ZnO particles in clinical dermatology settings.

Moreover, a separate clinical study involving a patient with actinic keratoses removal revealed significant improvements with ND-ZnO treatment. The patient, who had been suffering from persistent post-procedure burn, experienced notable healing after using cream comprising ND-ZnO particles for just one week. See FIG. 2D. The visual comparison between day 0 and day 7 demonstrated substantial reduction in burn persistence and overall skin recovery.

### Example 2: Collagen ELISA Assay

To assess the impact of ND-ZnO particles on collagen synthesis in HDFs, total collagen enzyme-linked immunosorbent assays (ELISAs) were employed. HDFs were initially seeded in 6-well plates, each well containing 2.5 mL of DMEM supplemented with 10% FBS. After a 72-hour culture period, or upon reaching 80% confluence, ND-ZnO particles at a concentration of 10 μL/mL, PBS, or a positive control comprising 100 μg/mL Vitamin C and 7 μg/mL Vitamin E was introduced for a further 72-hour coculture. Subsequently, collagen levels in the conditioned medium were quantified using an ELISA kit (Abcam), with absorbance measurements at 450 nm via a microplate reader.

To elucidate ND-ZnO particles' regenerative influence on HDF collagen synthesis, cells were first exposed to UVA at an intensity of 9 J/cm² to artificially age the fibroblast, followed by the addition of test solutions. This set included a blank control (HDFs with regular cell culture media, no UVA treatment), a negative control (HDFs with regular media, UVA treatment), and an ND-ZnO group (HDFs with media and ND-ZnO particles, UVA treatment).

Conversely, to evaluate ND-ZnO particles' protective capacity, test solutions, including a positive control (HDF with media and Vitamins C & E, UVA treatment), were added to each well prior to UVA exposure at 9 J/cm².

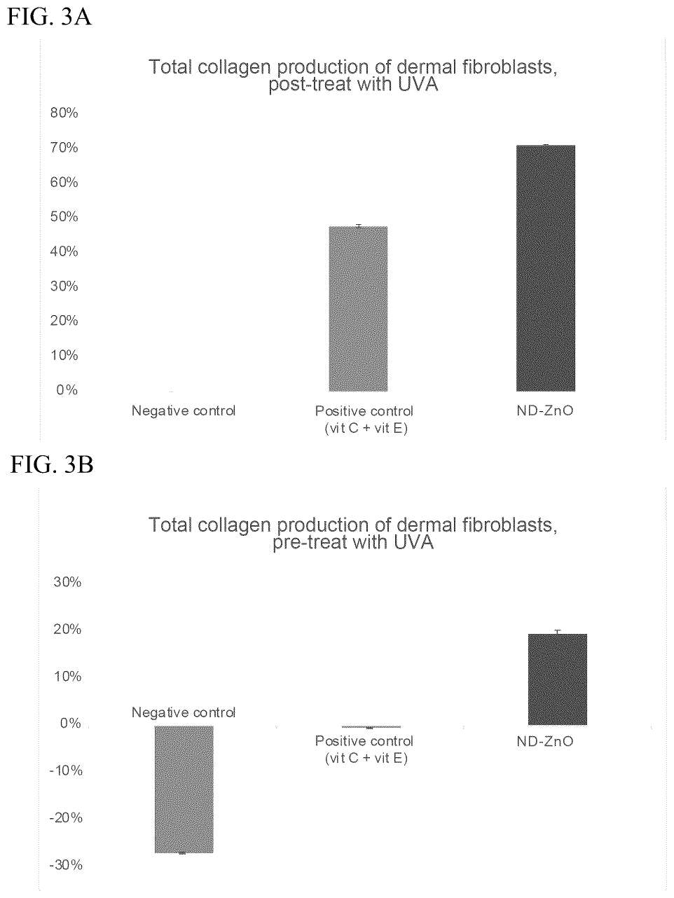

Both the UVA pre- and post-treatment ELISA assay sets demonstrated that ND-ZnO particles effectively prevented and reversed age-associated HDF collagen degradation more efficiently than all other treatment groups. In the UVA pre-treatment analysis (FIG. 3A), coculture with ND-ZnO particles restored 71.3% of aged HDF's collagen production capacity in comparison to the negative control (SD=0.31%, p<0.001).

In the UVA post-treatment evaluation (FIG. 3B), ND-ZnO not only defended HDFs against all UVA-induced collagen synthesis diminution but also improved HDF collagen synthesis by an average of 19.4% compared to the unaged blank control cells (SD=2.13, p<0.0001). In comparison to HDFs in the negative control group, which lost 27.1% of their collagen production capability, ND-ZnO-treated HDFs demonstrated 46.5% greater collagen production (p<0.0001). ND-ZnO particles performed significantly better than the Vitamin C & E positive control group in preventing age-induced collagen loss, despite the concentration of the Vitamin C & E solution being ten times that of the ND-ZnO particles solution.

The data indicates that ND-ZnO particles are highly effective in stimulating collagen production in aged HDFs, both in preventing collagen degradation and in promoting collagen production restoration in aged cells. This significant improvement in collagen synthesis highlights the potential of ND-ZnO particles as a powerful agent in skin regeneration therapies, offering superior protection and restoration of collagen production capabilities in dermal fibroblasts.

### Example 3: Reactive Oxygen Species Eradication Assay

Intracellular reactive oxygen species (ROS) production to evaluate ROS scavenging potential of ND-ZnO particles in response to irradiation was also investigated using EpiDerm Skin Model EPI-212-X tissues and 2,7-dichlorofluorescein diacetate (DCFH-DA), which enters cells passively and reacts with ROS to produce the highly fluorescent compound dichlorofluorescein (DCF). A working solution of DCFH-DA in DMSO was diluted in PBS and ND-ZnO and generic zinc oxide dispersions were created. After exposure to natural light at room temperature, fluorescence was recorded at intervals spanning 6 hours using the same plate reader parameters. See FIG. 1D.

### Example 4: Niacinamide Transdermal Delivery and Skin Permeation Assay

A diffusion cell was prepared by inserting a magnetic stirrer and placing reconstructed human epidermis tissue in the middle. For the setup, 7.5 mL of 1×PBS buffer with 4% BSA was added to the bottom section of each cell, and the sampling ports were sealed with parafilm. The experiment involved a set of three parallel cells. The water bath's temperature was maintained at 37° C. Prior to the introduction of the samples, the diffusion cell was pre-warmed for 10 minutes. Then, either an ND-ZnO and 5% niacinamide solution (2 mg/cm²) or a 5% niacinamide solution (2 mg/cm²) was added to the top section of each cell, with the final mass of approximately 3.53 mg or 3.53 μL, respectively. At intervals of 1, 2, 4, 8, 12, and 24 hours, 500 μL of reaction solution was drawn from each cell for analysis, and the cell was immediately refilled with the same volume of PBS buffer with 4% BSA. The drawn samples underwent a series of processing steps involving dilution with methanol, vortexing, centrifugation, and filtration using 0.22 μm PES syringe filters for supernatant collection.

For the calibration sample preparation, a standard solution of 1.0 mg/mL niacinamide in 1×PBS buffer with 4% BSA was prepared. Each calibration solution underwent a processing protocol like that of the permeation test samples, which involved removing BSA to prepare for quantification.

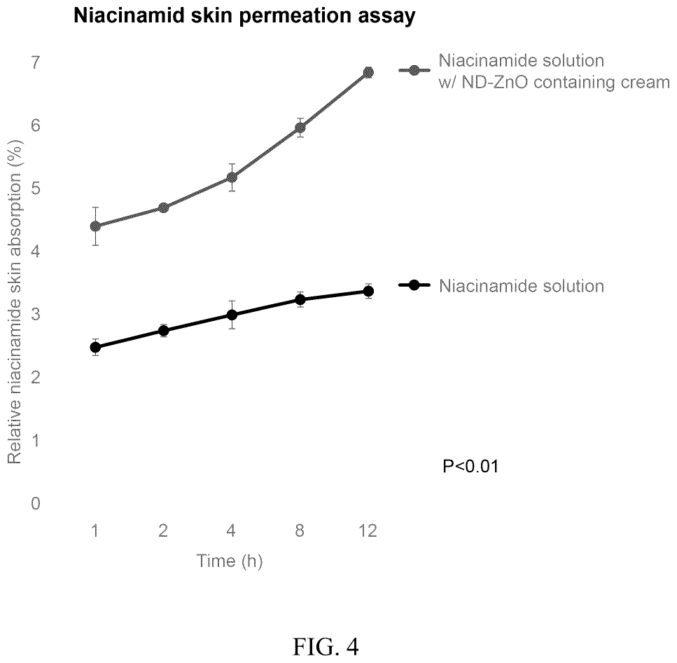

The results of the niacinamide skin permeation assay demonstrated that the ND-ZnO+5% niacinamide solution significantly enhanced niacinamide absorption compared to the niacinamide solution alone. See FIG. 4. Over a 12-hour period, the ND-ZnO containing cream achieved an average relative skin absorption of 6.85% (SD=0.086), while the niacinamide solution alone achieved 3.37% (SD=0.12), indicating a 203% increase in absorption.

This substantial improvement in skin absorption underscores the potential of ND-ZnO technology in maximizing the transdermal delivery of active agents like niacinamide. By enhancing the skin penetration of niacinamide, ND-ZnO particles not only improve the efficacy of niacinamide but may also achieve a synergistic effect when combined with other bioactive compounds, amplifying efficacy of topical skincare agents.

1-2. (canceled)

3. A method of promoting dermal fibroblast migration or growth in a subject comprising the step of applying a topical composition to the subject's skin, wherein the topical composition comprises particles with a core-shell structure, wherein the core comprises nanodiamond and the shell comprises zinc oxide.

4-11. (canceled)

12. A method of promoting collagen production in a subject comprising the step of applying a topical composition to the subject's skin, wherein the topical composition comprises particles with a core-shell structure, wherein the core comprises nanodiamond and the shell comprises zinc oxide.

13. The method according to claim 12, wherein the particles stimulate collagen production in dermal fibroblast cells.

14. The method according to claim 13, wherein the dermal fibroblast cells are aged dermal fibroblast cells.

15. The method according to claim 12, wherein collagen production is promoted after exposure of the subject's skin to ultraviolet (UV) radiation.

16. The method according to claim 15, wherein the UV radiation is UVA radiation.

17. The method according to claim 12, wherein the topical composition is applied at night.

18. A method of reducing collagen degradation in a subject, comprising the step of applying a topical composition to the subject's skin, wherein the topical composition comprises particles with a core-shell structure, wherein the core comprises nanodiamond and the shell comprises zinc oxide.

19. The method according to claim 18, wherein the collagen degradation is age-associated collagen degradation.

20. The method according to claim 18, wherein the collagen degradation is caused by exposure of the subject's skin to ultraviolet (UV) radiation.

21. The method according to claim 20, wherein the UV radiation is UVA radiation.

22-25. (canceled)

26. The method according to claim 12, wherein the topical composition further comprises at least one topically acceptable carrier.

27. The method according to claim 12, wherein the topical composition comprises 0.01 to 30 percent by weight of the particles.

28. The method according to claim 12, wherein the topical composition comprises between 1 μg/mL and 100 μg/mL of the particles.

29. The method according to claim 28, wherein the topical composition comprises 10 μg/mL of the particles.

30. The method according to claim 12, wherein the core comprises a nanodiamond having a diameter between 3 nm and 50 nm.

31. The method according to claim 30, wherein the core comprises a nanodiamond having a diameter between 3 nm and 10 nm.

32. The method according to claim 12, wherein the diameter of the particles is at least 100 nm.

33-34. (canceled)

35. The method according to claim 12, wherein the subject is a human subject.

36. The method according to claim 35, wherein the human subject is at least 25 years old.
